(19) United States
(12) Patent Application Publication
(10) Pub. No.: US 2005/0245935 A1
(43) Pub. Date: Nov. 3, 2005

(54) Surgical saw blade

(76) Inventors: Conor P. Casey, Ardagh (IE); Gordon Bryan, Waterfall (IE); David Tallon, Midleton (IE)

Correspondence Address: FLYNN, THIEL, BOUTELL & TANIS, P.C., 2026 RAMBLING ROAD, KALAMAZOO, MI, 49008-1631, US

(21) Appl. No.: 11/115,067

(22) Filed: Apr. 26, 2005

Related U.S. Application Data
(60) Provisional application No. 60/566,553, filed on Apr. 29, 2004.

(51) Int. Cl. A61B 17/32

(52) U.S. Cl. 606/82

(57) ABSTRACT

A two legged surgical saw blade comprises a base engagable with a chuck on a tool, a rigidified intermediate portion, and a lightened distal portion comprising spaced toothed legs and a spacer of reduced mass.

This application claims the benefit of U.S. Provisional Application No. 60/566 553, filed Apr. 29, 2004.

### FIELD OF THE INVENTION

This invention relates to a surgical saw blade, and particularly one of the types having a pair of parallel toothed edges spaced apart at its distal end.

### BACKGROUND OF THE INVENTION

In one prior blade of the aforementioned kind, two plates are fixed together at their proximal end portions to form the blade base, which is configured to be received in a suitable chuck on a powered handpiece. In an intermediate part of the saw blade, the toothed plates are bent away from each other at right angles and after a short distance laterally are bent again at right angles to form planar legs extending distally in respective parallel planes spaced symmetrically on opposite sides of and parallel to the blade base. The distal ends of the plates are toothed. The two legs have similar, but oppositely directed, elongate keyhole shaped slots therethrough between the step formed by the aforementioned bends and the toothed distal ends. The wide, circular end of the keyhole slots in the respective legs are longitudinally offset from each other and from the narrowed portions of the keyhole slots, so as to hold captive, but allow a range of longitudinal sliding displacement, of a cylindrical spacer.

In use such a blade is chucked in a power tool chuck and driven so that the tooth sets oscillate from side to side. Such blades may be used for dissection of bone from the pelvis or elsewhere in a patient, with the resulting bone fragment being usable to repair bone damage elsewhere in the body (e.g. for insertion into the spinal column). Such blades, particularly in relatively forceful use by a surgeon, may experience unwanted flexing in the area of the aforementioned right angle bends, allowing the legs to shift relative to each other and thus allow their toothed ends to shift with respect to each other and out of their intended mutual position of use, thereby interfering with cutting.

Lengthening of the legs increases the mass of the blade, particularly at and near its distal end, such that, during oscillation, the increased momentum of the swinging distal portion of the blade may result in an unwelcome increase in vibration of the handpiece in the hands of the surgeon, and indeed make it more difficult for the surgeon to perform a resection without additional trauma to the patient.

As described, the saw blade is chucked in a surgical power tool and driven so that the tooth sets oscillate from side to side. It is common for new generation surgical power tools to run at higher speeds than their predecessors and so blades may be driven at higher speeds than in the past. The faster operating speeds (higher oscillation frequency) can present problems for prior blades.

For example, in prior blades of this type, as the frequency of oscillation increases and approaches the natural resonant frequency of the blade, the blade may resonate (vibrate). The vibration may be normal (substantially perpendicular) to the plane of oscillation or may be torsional approximately about the central axis of the blade or it may be a combination of normal and torsional. In prior blades, the severity of the vibration may be such that the out of plane movement of the distal end of the blade is quite visible to the naked eye and in some cases the vibration may be so severe as to lead to blade breakage.

### SUMMARY OF THE INVENTION

A two legged surgical saw blade comprises a base engagable with a chuck on a tool, a rigidified intermediate portion, and a lightened distal portion comprising spaced toothed legs.

### BRIEF DESCRIPTION OF THE DRAWINGS

FIG. 1 is a pictorial view of a saw blade embodying the invention and schematically showing a compatible chuck and handpiece.

FIG. 2 is a pictorial view of the FIG. 1 saw blade taken from a different point of view.

FIG. 3 is an elevational view of a blank usable in forming the FIG. 1 saw blade.

FIG. 4 is a side view of the FIG. 3 blank.

FIG. 5 is a side view of one unassembled part of the FIG. 1 blade.

FIG. 6 is an elevational view of the FIG. 5 part.

FIG. 7 is a side view of another unassembled part of the FIG. 1 blade.

FIG. 8 is an elevational view of the FIG. 7 member.

FIG. 9 is an elevational view of an assembled FIG. 1 blade.

FIG. 10 is a side view of the FIG. 9 blade, schematically showing same in a position of use.

FIG. 11 is an enlarged central cross sectional view of the spacer of the FIG. 1 blade.

FIG. 12 is a pictorial view similar to FIG. 2 but showing a modification.

FIG. 13 is a pictorial view of the FIG. 12 saw blade taken from a somewhat different point of view.

FIG. 14 is an elevational view of an assembled FIG. 12 blade.

FIG. 15 is a side view of the FIG. 14 blade.

FIG. 16 is an enlarged fragment of FIG. 15.

FIG. 17 is a pictorial view generally similar to FIG. 1 and showing a modification.

FIG. 18 is a view similar to FIG. 17 with the spacer relocated from its FIG. 17 distal position to its proximal position.

FIG. 18A is an elevational view of a blank useable in the forming FIG. 17 saw blade.

FIG. 19 is an elevational view of an assembled FIG. 17 blade, showing the spacer in its distal position.

FIG. 20 is an elevational view similar to FIG. 19, but showing the spacer in its proximal position, corresponding to FIG. 18.

FIG. 21 is a side view of the FIG. 19 blade.

FIG. 22 is a side view of the FIG. 20. blade.

FIG. 23 is an enlarged elevational view of a blank useable in forming the brace of the FIG. 17 blade.

FIG. 24 is a central cross sectional view substantially taken on the line 24-24 of FIG. 23.

FIG. 25 is an elevational view of the brace of the FIG. 17 blade, formed from the FIG. 23 blank.

FIG. 26 is a central cross sectional view substantially taken on the line 26-26 of FIG. 25.

FIG. 27 is an enlarged elevational view of the spacer of the FIG. 17 blade.

### DETAILED DESCRIPTION

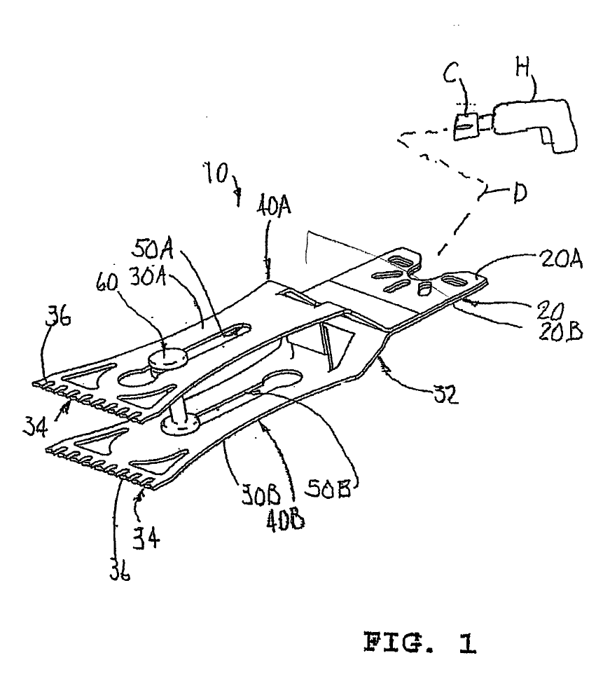

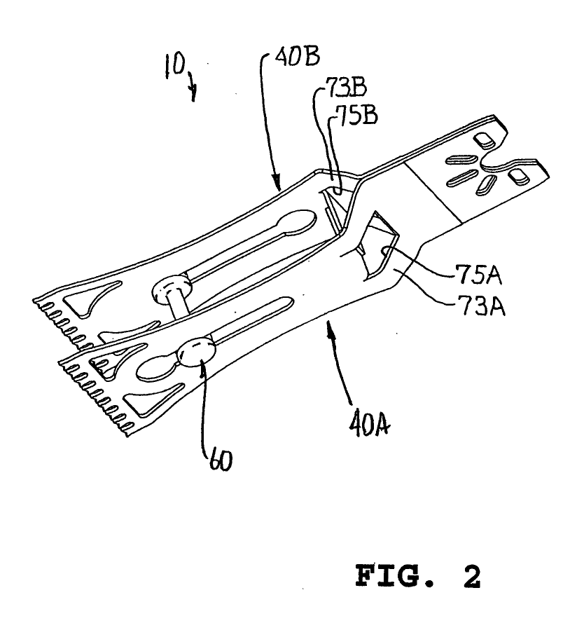

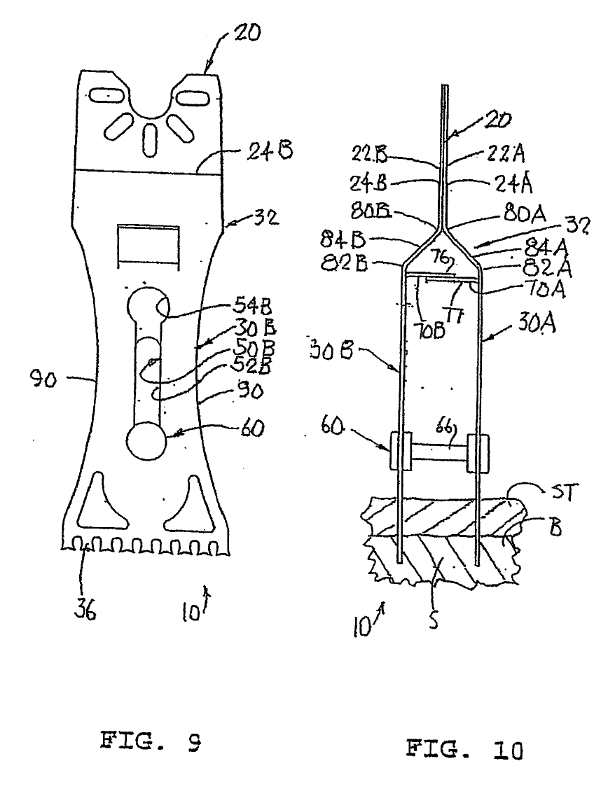

A preferred embodiment of the invention comprises a surgical saw blade 10 (FIGS. 1 and 2) of Casper type, namely of the type having a pair of parallel toothed edges spaced apart at the distal end thereof for removing a slice S (FIG. 10) of hard tissue, such as bone B.

The blade 10 is preferably constructed of flat, substantially rigid stock, typically of surgical grade, rigid, stainless steel sheet. The blade 10 (FIG. 1) has a proximal portion defining a base 20 configured, as desired, for reception and driving by the chuck C of a suitable, powered, surgical handpiece H, as schematically indicated by the dotted line D. The chuck C and the corresponding configuration of the base 20 here shown of conventional type. The blade base 20 is here configured in the manner shown in U.S. Design Patent 343 247, issued Jan. 11, 1994 and assigned to Stryker Corporation, the Assignee of the present invention. Correspondingly, chuck C and handpiece H in the present example may be of the type available from present Assignee Stryker Corporation, located in Kalamazoo, Mich. U.S.A., as Model Number 5400-34.

The blade 10 further includes an opposed pair of substantially parallel generally planar legs 30A and 30B which extend distally from an intermediate portion 32 of the blade 10, from which the base 20 extends proximally. The distal edges 34 of the blade legs 30A and 30B are preferably parallel, are preferably identically configured, and have material removal structure, here in the form of a series of saw teeth 36. The teeth 36 may be conventional and of any desired configuration.

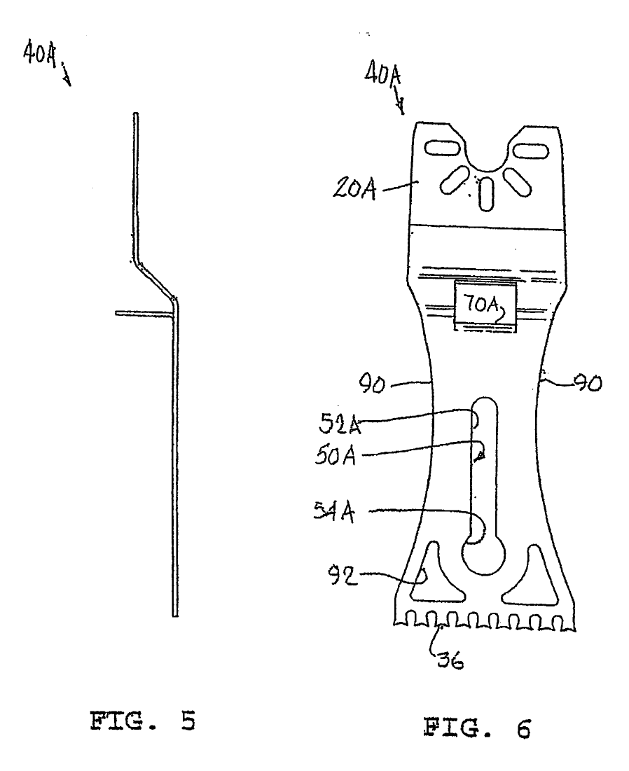

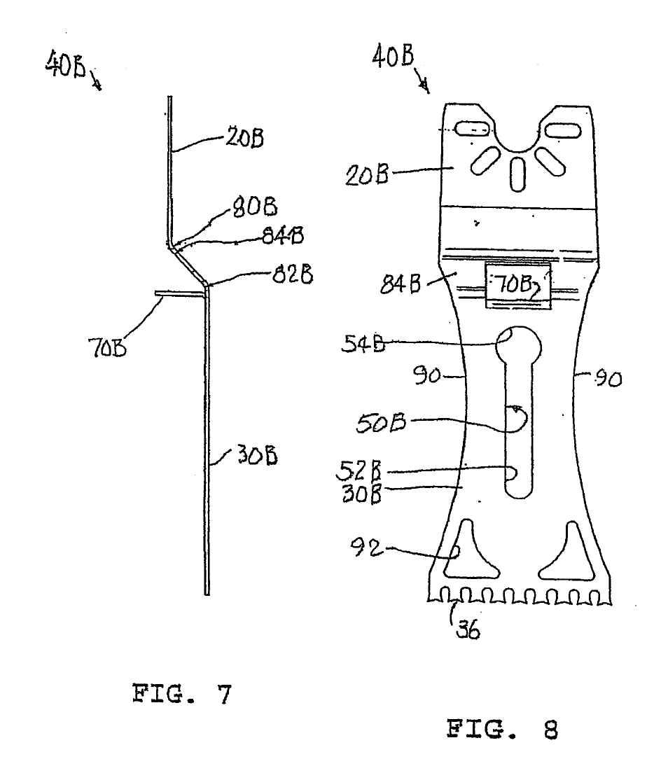

It is convenient to form the blade 10 from an opposing pair of plate-like blanks of rigid sheet or plate material, one such blank being shown at 40B in FIG. 3. The intermediate portion 32B of the blank 40B is suitably deformed (e.g. by bending, curving or the like), to offset the leg (here the leg 30B) to a plane spaced from and preferably substantially parallel to the plane of the base portion 20B of the blank 40B. By similarly configuring a second blank 40A, as seen in FIGS. 1 and 10, the corresponding base portions 20A and 20B thereof can be placed in face-to-face mapping relation, as shown in FIGS. 1 and 10, and then fixed together in a conventional way, as by brazing, silver soldering, adhesive bonding, welding or the like, with the legs 30A and 30B spaced on opposite sides of the plane of the base 20. The thus opposable blanks 40A (FIGS. 5 and 6) and 40B (FIGS. 7 and 8) comprise the two major structural members of the blade 10.

As seen in FIGS. 1, 2, 6 and 8, the blanks 40A and 40B are generally similar, though not identical. For example, while each of the legs 30A and 30B has, extending along the central longitudinal axis thereof, a keyhole shaped slot (at 50A and 50B respectively), wherein the elongate narrow end portions (at 52A and 52B, respectively) of the slots are directly opposed, the keyhole slots 50A and 50B have their circular widened ends (at 54A and 54B, respectively) longitudinally spaced from each other, adjacent the distal and proximal ends, respectively, of the legs 30A and 30B. Indeed, the widened ends 54A and 54B are longitudinally spaced beyond and hence do not oppose any part of the opposite slot 50B and 50A (see for example FIGS. 1 and 9).

An elongate spacer 60 (FIGS. 1, 10 and 11) is preferably of circular cross section and has opposed end portions 62, each having an annular groove 64. The axial width of the annular grooves 64 slightly exceeds the thickness of the leg portions 30A and 30B. The outside diameter of the end portions 62 exceeds the width of the narrow end portions 52A and 52B of the keyhole slots 50A and 50B. The diameter at the inner circumference of the annular grooves 64 is less than the width of the narrow end portions 52A and 52B. The outside diameter of the spacer end portions 62 is less than the diameter of the circular widened portions 54A and 54B of the keyhole slots 58A and 58B.

As a result, prior to fixing together of the blanks 40A and 40B, the blanks can be opposed and displaced longitudinally with respect to each other to allow insertion of the spacer end portions 62 into the widened keyhole slot portions 54A and 54B, with the annular grooves 64 of the spacer 60 respectively in the planes of the blanks 40A and 40B. Given that, displacement of the blanks 40A and 40B with respect to each other, in a direction to achieve the positioning of FIG. 1, displaces the spacer 60 longitudinally of the legs 30A and 30B, from the widened ends 54A and 54B of the keyhole slots and into the elongate narrow end portions 52A and 52B thereof, with the edges of the elongate narrow end portions 52A and 52B slidably engaged in corresponding ones of the annular grooves 64 of the spacer 60. Thus, with the base portions 20A and 20B of the respective blanks 40A and 40B mappingly opposed and fixed together face-to-face, as by means such as those suggested above, the spacer 60 is held captive in the elongate narrow end portions 52A and 52B of the keyhole slots 50A and 50B. The spacer prevents the legs 30A and 30B of the blade 10, and particularly distal portions thereof, from flexing toward and away from each other, so as to maintain the toothed distal edges 34 of the legs 30A and 30B fixed relative to each other.

Turning now to additional features of the disclosed saw blade 10, the intermediate portion 32B of the blank 40B includes a tab 70B, here of generally rectangular form, defined by a generally U-shaped line of separation 72. Whereas the tab 70B (FIG. 10) is preferably rectangular in shape, a tapered and/or curved edge tab is also contemplated. The tab 70B is preferably of lateral width equal to or somewhat less than the half adjacent width of the corresponding leg, for example 35-50% of such width. The tab 70B is bendable out of the plane of the blank 40B generally along a widthwise dashed line 74B between the free ends of the line of separation 72, and near the proximal end of the keyhole slot 50B, leaving a residual hole 75B in the blank.

As seen in FIGS. 7 and 8, the blank 40B is bent at locations 80B and 82B to offset the leg 30B to a plane spaced from and substantially parallel with that of the base portion 20B. The bend lines 80B and 82B extend widthwise of the blank 40B and are substantially parallel to each other and spaced by a laterally extending, angled ramp 84B.

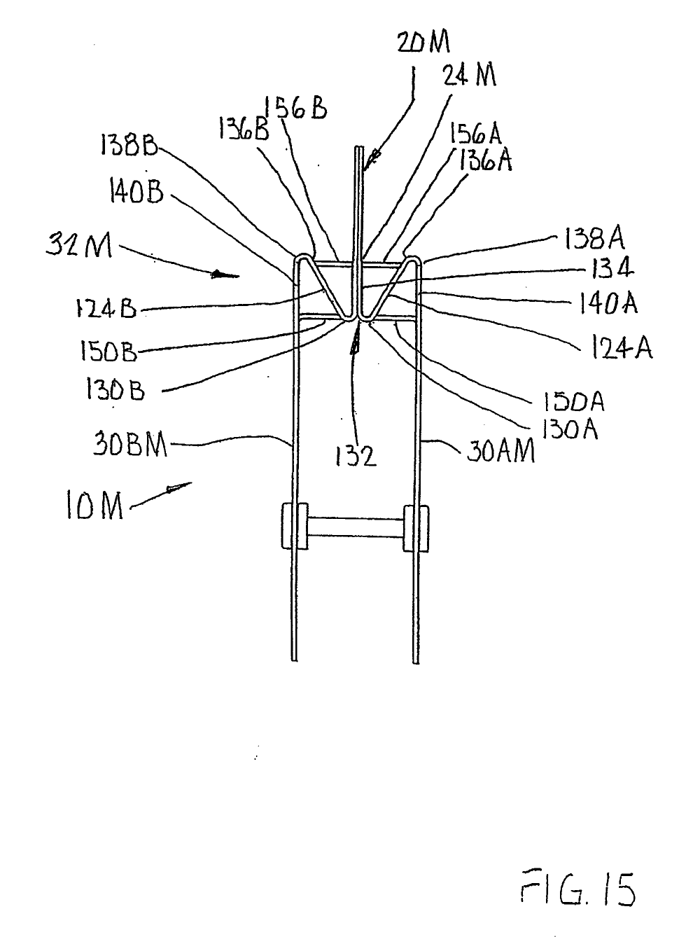

The ramp 84B (FIG. 7) is preferably angled at about 45° to both the base portion 20B and leg 30B, at the bends 80B and 82B. Although the last mentioned angle is preferred, it is contemplated that the angles of the bends 80B and 82B may be varied between 30 and 60°, though in such a way as to maintain the base portion 20B and leg 30B substantially parallel. Indeed, a given said bend angle may be increased even more, as discussed below with respect to the modification shown in FIGS. 12-16. The tab 70B is seen in FIG. 7 to bend, preferably at about 90°, from the plane of the leg 30B, so as to oppose, in the longitudinal direction of the blank 40B, and be close spaced distally from the ramp 84B. As seen in FIG. 7, the tab 70B extends from the leg 30B to a distance beyond the plane of the base portion 20B and so is somewhat longer than, but not twice as long as, the offset between the planes of the base portion 20B and leg 30B.

The blank 40A includes a generally similar tab 70A, residual hole 75A and ramp 84A.

In the embodiment shown, the outboard faces 22A and 22B (FIG. 10) of the base portions 20A and 20B each have a laterally extending, proximally facing step 24A and 24B, respectively. Thus, the base 20 of the assembled saw blade 10 is thinner proximally of the steps 24A and 24B and thicker distally of steps. The reduced thickness of the proximal portion of the base 20 may be provided to facilitate entry into the chuck C (FIG. 1), without reducing the rigidity of the rest of the blade 10.

With the saw blade 10 assembled as in FIG. 10, the tabs 70A and 70B overlap at 76 and are there fixedly connected by any convenient means such as brazing, silver soldering, welding, etc. to form a bridge 77 rigidly connecting the proximal end portions of the legs 30A and 30B substantially at right angles thereto, and adjacent the laterally extending bend lines 82A and 82B. As seen in FIG. 10, the bridge 77 and ramps 84A and 84B form a generally triangular profile which strongly resists bending or twisting of the saw blade 10, and in particular resists mispositioning of the legs 30A and 30B with respect to each other and to the base 20. This triangular arrangement assists the spacer 60 in maintaining the legs 30A and 30B in their intended planes spaced on opposite sides of the plane of the base 20.

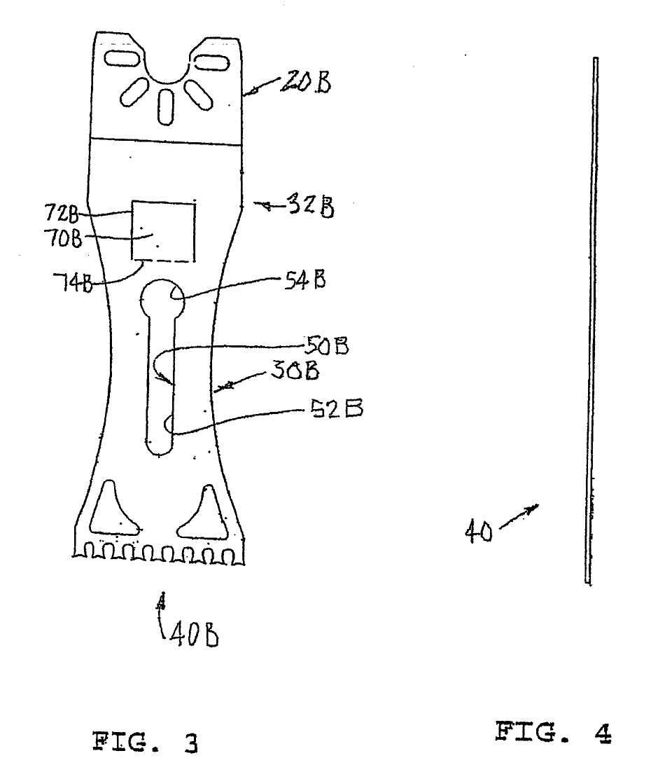

In this way, particularly the legs 30A and 30B of the saw blade 10, can maintain their intended shape and relative location shown in FIG. 10, despite longitudinal pushing, twisting, rocking, and side-to-side etc. forces applied by the surgeon to the handpiece H carrying the saw blade 10, during cutting. This becomes even more important in saw blades which are longer and/or wider than the example shown in FIGS. 1-10.

In addition, the saw blade 10 embodying the invention is configured to reduce, or minimize, weight, particularly in the distal portion thereof including the legs 30A and 30B and spacer 60, without impairing the rigidity of the saw blade 10. Thus, in the embodiment shown, the width of the legs 30A and 30B is reduced toward the longitudinal central portions thereof, here by concavely shaping the profile of the longitudinal side edges 90 of the legs 30A and 30B, as seen for example in FIGS. 6, 8 and 9. The side edges 90 are preferably smoothly curved to minimize sharp corners or edges, which might weaken the saw blade 10 or otherwise interfere with its convenient use. The reduction in width of the legs 30A and 30B preferably begins in the area of the ramps 84A and 84B and extends distally almost to the teeth 35. The legs 30A and 30B are narrowest generally in the middle of that range, adjacent the narrow end portions 52A and 52B of the keyhole slots 50A and 50B, and wider in the region of the widened ends 54A and 54B of such keyhole slots, so as to maintain a sufficient width of material to maintain the strength and rigidity of the legs 30A and 30B.

The distal portion of the legs 30A and 30B is further lightened by holes 92. The holes 92 here have sides respectively spaced from the leg side edges 90, the bases of the teeth 36 and the adjacent end portion of the corresponding keyhole slots 50A and 50B. The result of substantially evenly spacing the edges of each hole 92 from the mentioned structural features is a hole shape that is roughly triangular, the corners of the hole being rounded to relieve stress, and the inboard edges of the holes 92 preferably being curved to complement the adjacent shape of the narrow end portion 54A in the blank 40A. For ease of manufacture, the lightening holes 92 in the blank 40B are similarly configured.

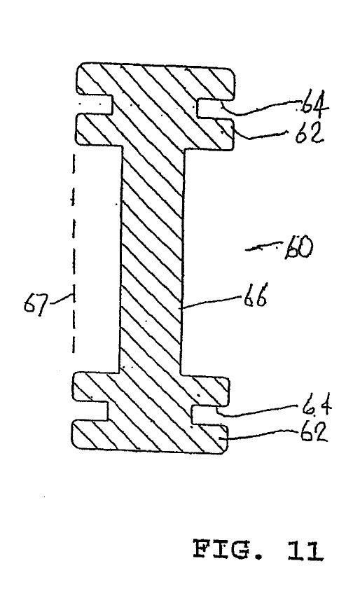

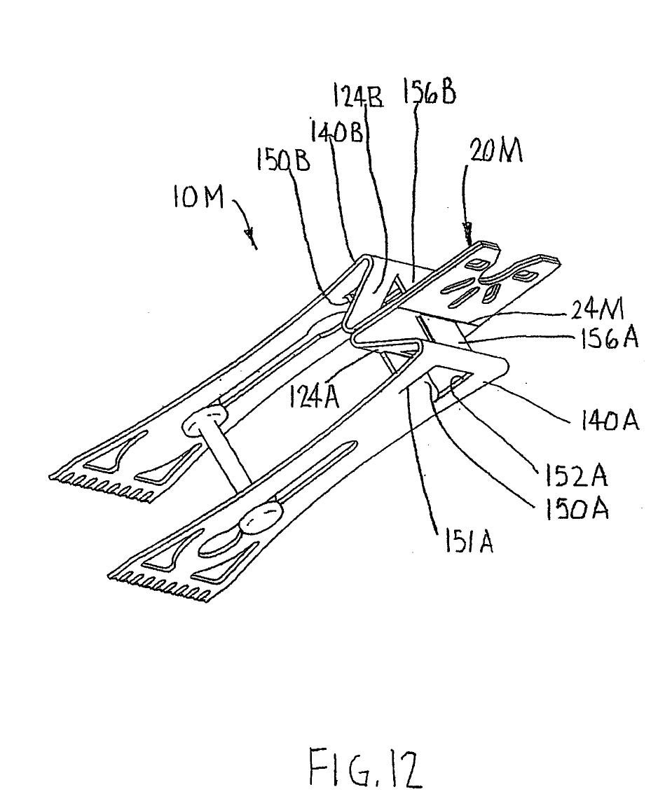

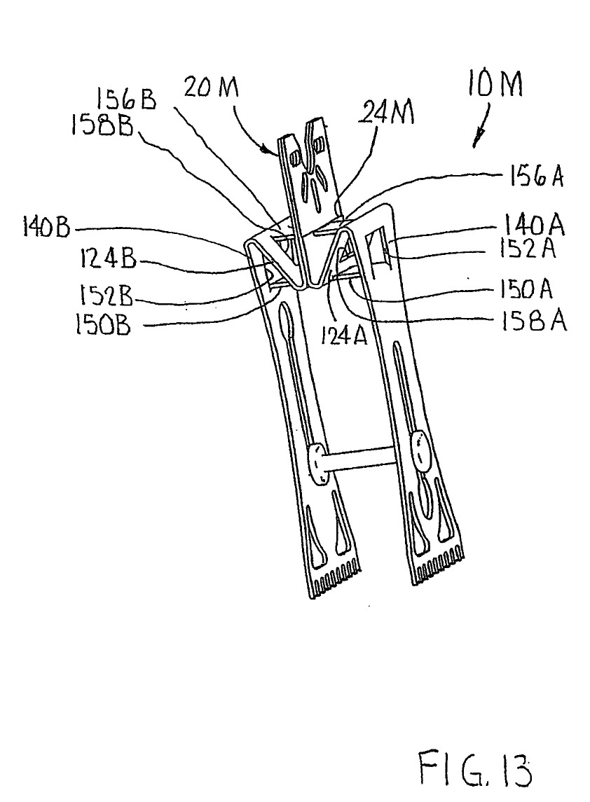

Further, the spacer 60 has an elongate midportion 66 of substantially reduced diameter, in the embodiment shown less than the diameter measured between the radially inner faces of either annular groove 64. Thus, in the embodiment shown, the spacer 60 is of generally dumbbell shape, with relatively large diameter, annularly grooved, end portions 62 spaced by an elongate, substantially cylindrical, and substantially thinner midportion. This eliminates the weight of material immediately outboard of the midportion 66 and radially inboard of the imaginary cylinder defined by the outer periphery of the end portions 62 and indicated by the dotted line 67 in FIG. 11. This eliminates a substantial portion of the weight that is present in conventional cylindrical spacers used in prior saw blades of this general type.

By minimizing the mass of the legs 30A and 30B and spacer 60, which define the distal part of the blade 10 and during oscillation have the widest swing, or travel, the vibration experienced by the surgeon holding and operating the handpiece H is reduced, as is any tendency of the saw blade to jump about during cutting, thereby in turn reducing the risk of inadvertent, unwanted, damage to tissue in the neighborhood of the cut being made. In the embodiment shown, the tabs 70A and 70B are slightly, but sufficiently, displaced longitudinally of the saw blade 10 to avoid interference therebetween and allow the snug overlapping fit therebetween shown in FIG. 10.

It is particularly important to note that the overlapping and fixedly connected tabs 70A and 70B, forming the bridge 77, so increase the stiffness of the blade assembly that the natural resonant frequency of the blade occurs at a higher frequency than the frequency of oscillation of the power tool and chuck, thus preventing, in contrast to above discussed prior art blades, the occurrence of resonance and resulting vibration of the inventive saw blade 10, even at the above described higher speeds (e.g. 25,000 cycles/minute) at which new generation power tools are intended to run.

Moreover, the lightening of the distal portion of the blade (e.g. as by the above discussed narrowed spacer midportion 66, concave blade side edges 90 and blade holes 92) tends advantageously also to increase the natural resonant frequency of the blade, and thus enhances the benefits above discussed.

### MODIFICATION

The FIGS. 12-16 modified blade 10M is preferably similar to the FIGS. 1-11 blade above described, except as hereafter discussed.

Parts of the FIGS. 12-16 blade 10M, corresponding to parts of the FIGS. 1-11 blade 10, will carry the same reference characters but with the added suffix M.

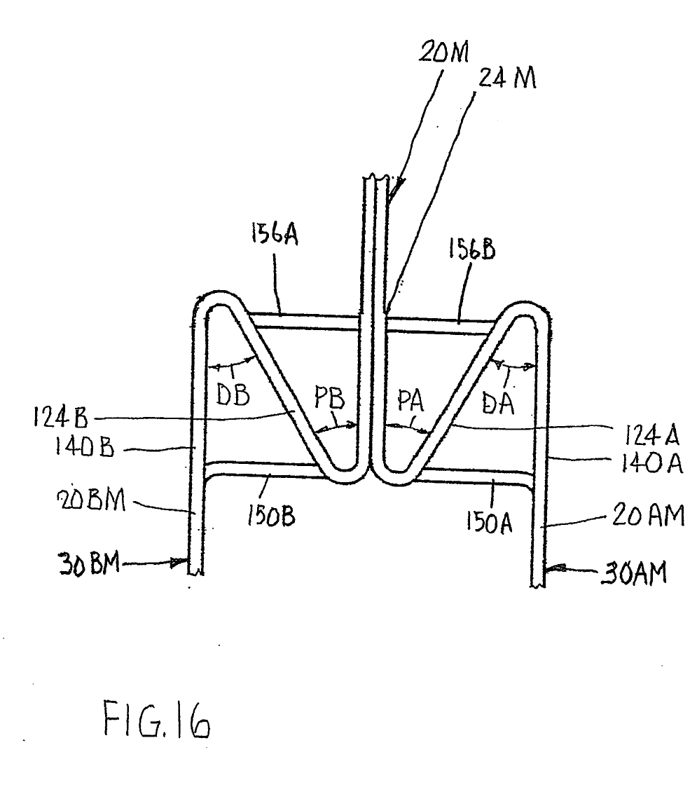

The intermediate portion 32M of the blade 10M comprises a bilaterally symmetrical profile which, as seen in FIGS. 15 and 16, comprises laterally opposed ramps 124A and 124B (1) with laterally adjacent distal ends 130A and 130B fixed to the distal end 132 of a distal extension 134 of the base 20M and (2) angled laterally outwardly and proximally therefrom to laterally spaced proximal ends 136A and 136B. The latter are respectively fixed to the proximal ends 138A and 138B of respective proximal extensions 140A and 140B of the legs 30AM and 30BM, respectively. Thus, the distal extension 134 of the base 20M, the ramp 130A and the leg proximal extension 140A form substantially a Z-shape in profile. The base extension 134, ramp 124B and leg proximal extension 140B form substantially a Z-shape in profile which is opposed to and substantially the mirror image of the Z-shape 134, 124B, 140B. Alternatively, and ignoring the base 20M, the ramps 124A and 124B and the corresponding leg proximal extensions 140A and 140B may be seen to form substantially an M-shaped profile.

In one unit constructed according to the invention, the base 20M (like the base 20 of FIGS. 1-11) may comprise two fixed-together, face-to-face abutting, plate portions which at their distal ends fold proximally through an obtuse angle into the ramps 24A and 124B (FIG. 15), which in turn fold at an acute angle into the proximal extensions 140A and 140B of the legs 30AM and 30BM. It is convenient if the proximally opening angles PA and PB (FIG. 16) between the base 20M and flanking ramps 124A and 124B and the distally opening angles DA and DB between the ramps 124A and 124B and the leg proximal extensions 140A and 140B, respectively, are all the same, thereby maintaining the legs 30AM and 30BM parallel to each other and equally laterally spaced from and parallel to a central plane defined by the base 20M. The mentioned acute angles PA, PB, DA and DB may be any within a range of angles, such as from 15 to 50° (about 30° in the embodiment of FIG. 16).

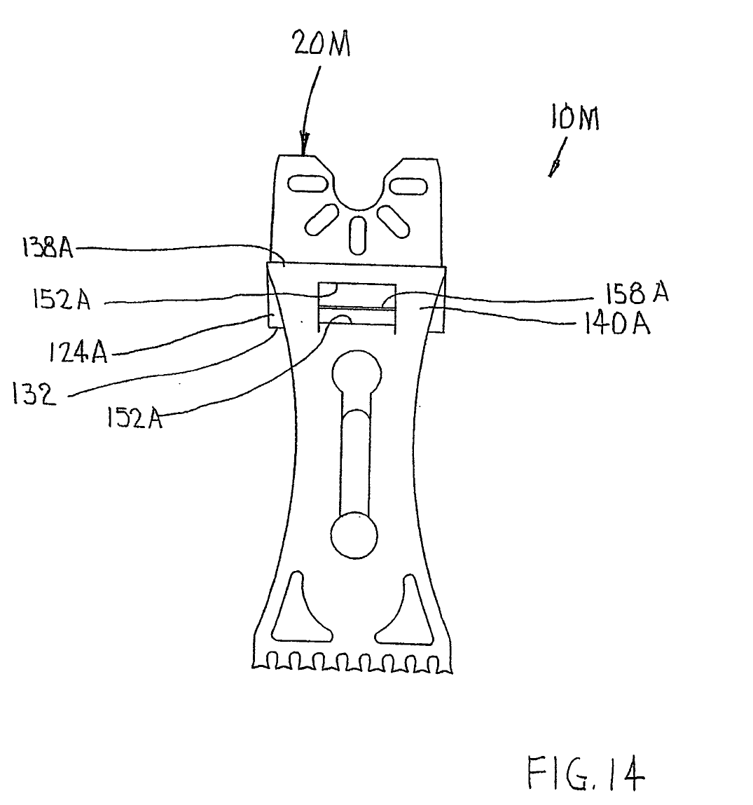

The distal tabs 150A and 150B extend laterally, preferably in a coplanar manner, generally toward each other, from their respective legs 30AM and 30BM to fixed engagement with the respective ramps 124A and 124B adjacent the respective distal ends 130A and 130B of the latter. Similarly, preferably coplanar, proximal tabs 156A and 156B extend laterally toward each other from their respective ramps 124A and 124B, adjacent the respective proximal ramp ends 136A and 136B, to fixedly engage the opposite sides of the base 20M. In the embodiment shown, the distal tabs 150A and 150B (FIG. 13) are bent out of the respective planes of their corresponding legs 30AM and 30BM substantially at right angles thereto.

The generally U-shaped line of separation 151A and the end portion of the tab 150A define a hole 152A of corresponding shape (here rectangular) through the corresponding leg proximal extension 140A. Preferably in the same manner, the bending of the opposed distal tab 150B leaves a corresponding hole 152B in the opposite leg 30BM.

It is convenient to similarly form the proximal tabs 156A and 156B. For example, the proximal tab 156A is bent laterally inward towards the blade base 20M, the corresponding line of separation 157A and the bent portion of such proximal tab 156A leaving a hole 158A in the proximal portion of the corresponding ramp 124A. Conveniently, the opposed proximal tab 156B is bent laterally inward from the plane of the corresponding ramp 124B to leave a hole 158B therein. In the embodiment shown, the resulting holes 152A, 152B, 158A and 158B are laterally opposed and at least partially overlap. The distal tabs 150A and 150B preferably are bent substantially at a right angle to the corresponding legs 30AM and 30BM and fold generally in a distal direction, whereas the proximal tabs 156A and 156B fold from their corresponding ramps in a generally proximal direction. The tabs 150A and 150B are preferably parallel to the corresponding tabs 158A and 158B all are preferably substantially perpendicular to the blade base 20M and legs 30AM and 30BM.

The free ends of the tabs 150A and 150B are fixed, by any convenient means such as welding, to the corresponding ramps 124A and 124B, and similarly, the free ends of the proximal tabs 156A and 156B are preferably fixed, by any convenient means such as welding, to the opposite sides of the blade base 20M, preferably at or somewhat distal of the step 24M.

The tabs 150A and 150B and attached, intervening, bent, distal portions of the ramps 124A, 124B define a rigid bridge 160 rigidly joining the proximal portions of the legs 30AM, 30BM. The result, as seen in FIGS. 15 and 16, is a diagonally braced, rigid, box structure connecting each legs 30AM and 30BM to the blade base 20M. This structure is intended to substantially provide the advantageously increased stiffness and resonant frequency above described with respect to the FIGS. 1-11 saw blade 10, although at the cost of more structural complexity than the FIGS. 1-11 saw blade 10.

### FURTHER MODIFICATION

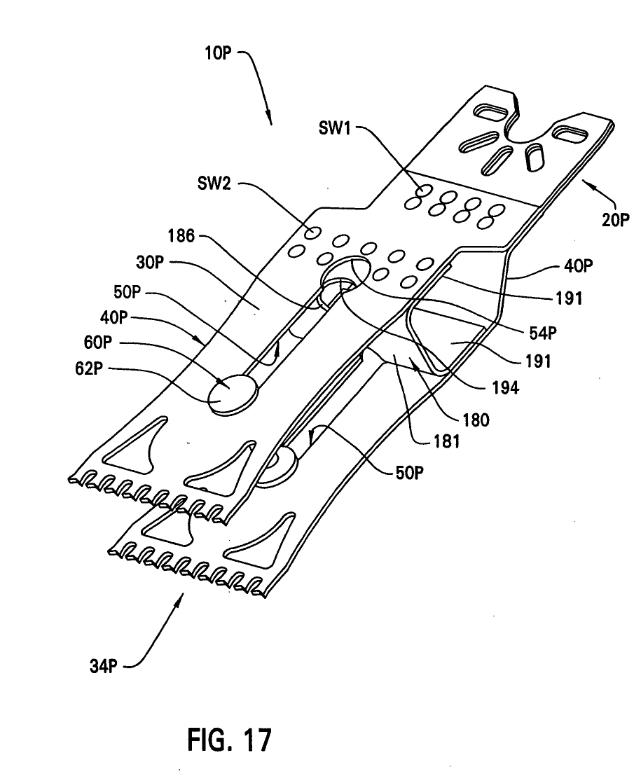

The FIGS. 17-27 modified blade 10P is preferably similar to the FIGS. 1-11 blade 10 above described, except as hereafter discussed.

Parts of the FIGS. 17-27 blade 10P, corresponding to parts of the FIGS. 1-11 blade 10, will carry the same reference characters but with the added suffix P.

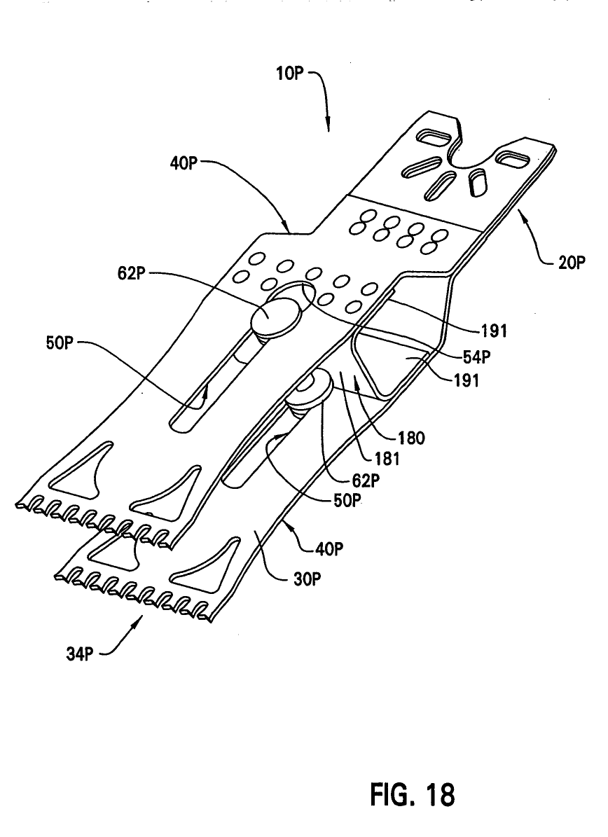

The blade 10P is conveniently formed from an opposing pair of flat plate-like blanks 40P (FIG. 18A) which blanks 40P are preferably identical to each other, unlike in the blades 10 and 10M above discussed, thereby reducing by a factor of two the patterns or dies which may be used to form such blanks 40P.

Unlike the FIG. 2 blank 40A, and generally like the blank 40B, of FIG. 2 the blanks 40P of FIG. 18A, for a given blade 10P, have their keyhole shaped slots 50P oriented with the circular widened end 54P disposed proximally of their elongate narrow end portions 52P.

As with the above described blades 10 and 10M, the two distal edges 34P of blanks 40P may vary in spacing from proximal blade end to distal blade end. By way of example, the distal edge 34P of the blank 40P is slightly wider than its proximal portion, located at the base 20P of the blade 10P.

In the assembled blade 10P (FIG. 21), generally as in the assembled blade 10 (FIG. 10), as above discussed, the opposed blanks 40, are bent,. preferably through about 45°, at transversely extending locations 80P and 82P, to offset the legs 30P to planes spaced from, substantially parallel to and preferably equidistant from the plane of the blade base portion 20P. Thus, the base portion 20P connects through angled ramps 84P to the legs 30P. The ramps 84P thus are in the blade intermediate portion 32P.

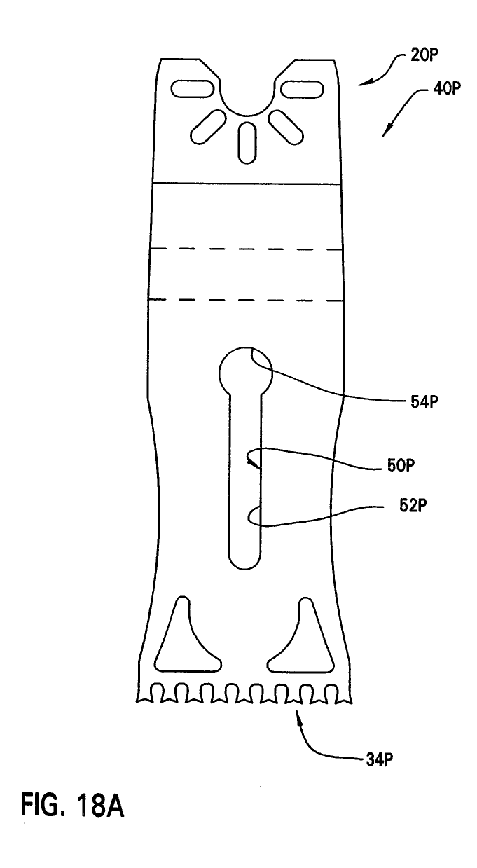

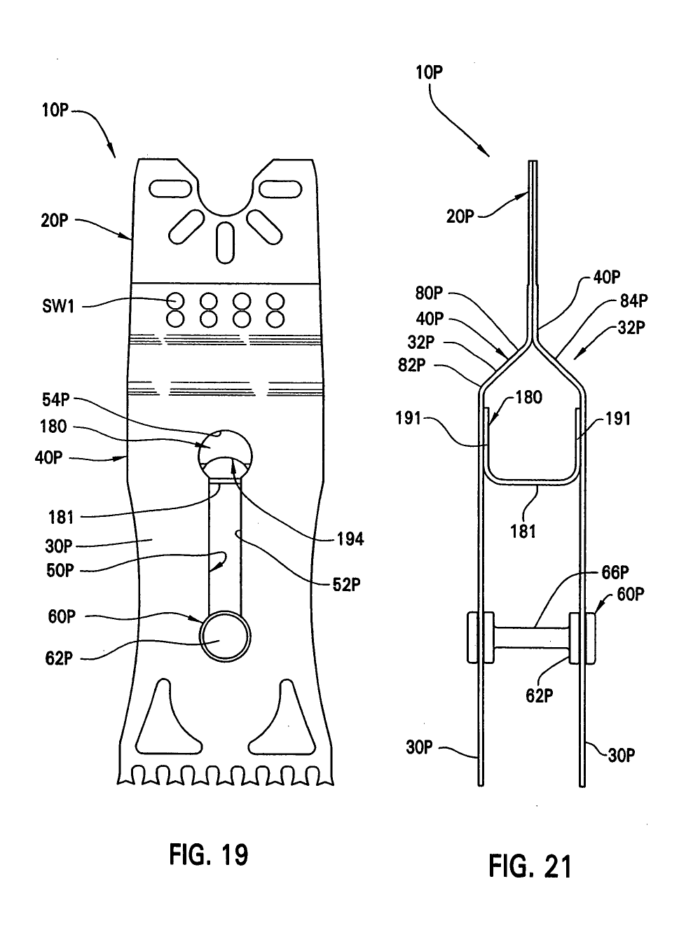

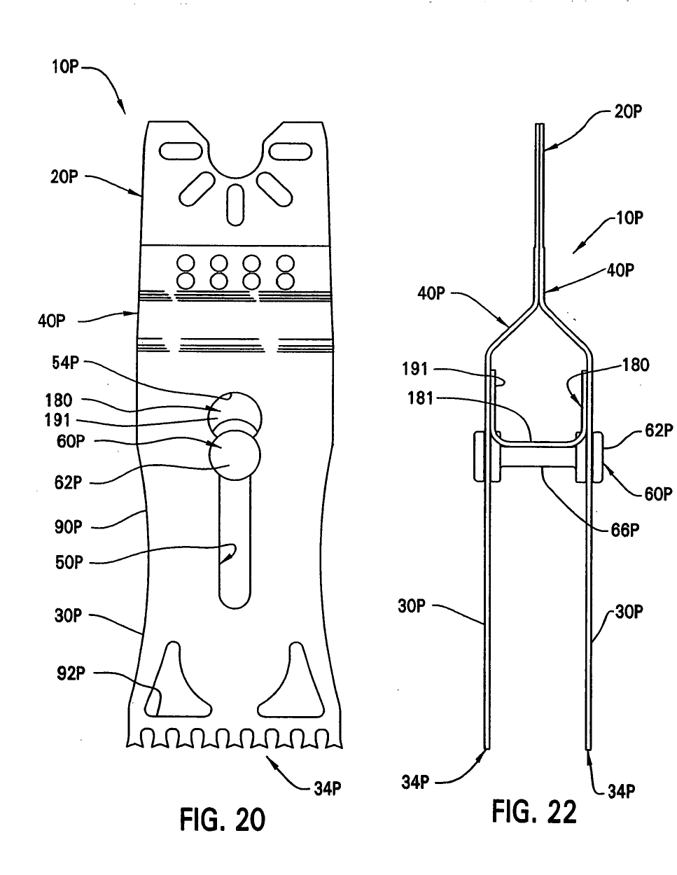

A substantially rigid bridge 180 (FIGS. 17 and 18) extends widthwise of the blade 10P and fixedly connects the opposed bent blanks 40P at the proximal portion of their legs 30P. In particular, the bridge 180 includes a web 181 extending fixedly between and perpendicular to the bent blanks 40P and extending transversely substantially the full width of the bent blanks 40P substantially at right angles to the opposite length edges of the blanks 40P and the central length axis of the blade 10P. The web 181 preferably is sufficiently distally spaced from the blade base 20P (FIGS. 20 and 22), and toward the two distal edge 34P of the blade 10P, as to interfere with and so define the proximal limiting location of the sliding spacer 60P. To allow location of the web 181 as far distally on the blade 10P as useable, the web 181 has reliefs 186 (FIGS. 19, 23 and 24) spaced transversely from each other and opening toward proximal portions of the keyhole slots 50P. The reliefs 186 are sized to receive the respective end portions 62P of the spacer 60P, such that the spacer's shaft-like midportion 66P in its proximal position abuts the distal face of the web 181 (FIG. 22).

To facilitate proper location and reliable fixation of the web 181 with respect to the opposed legs 30P, it is convenient to form the bridge 180 as a generally U, or channel, shaped cross section element of material similar to that of the blanks 40P, such as surgical grade stainless steel sheet. The spacer 60P is preferably also of surgical grade stainless steel.

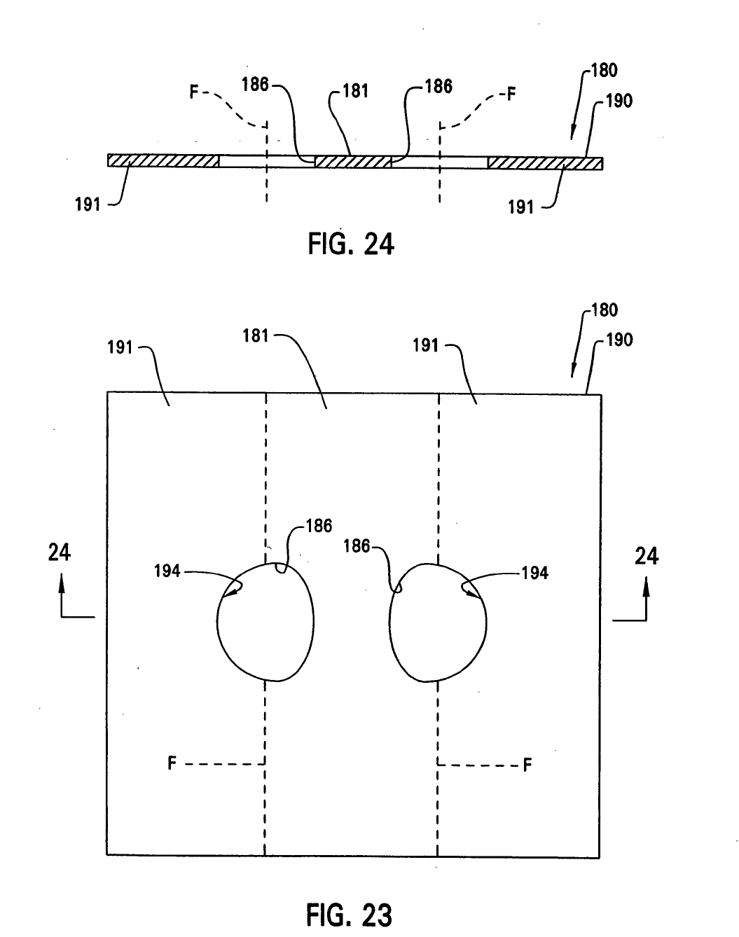

Thus, as seen in FIGS. 23 and 24, the bridge 180 may start as a generally rectangular blank of sheet material 190 whose central part defines the web 181 and is flanked by strips defining arms 191, separated therefrom by fold zones centered on the dotted lines F. The reliefs 186 are formed as somewhat flattened, adjacent sides of laterally spaced, through holes 194, which are otherwise of generally circular shape. The central portion of the fold zones, generally indicated by the dotted lines F, extend through the holes 194.

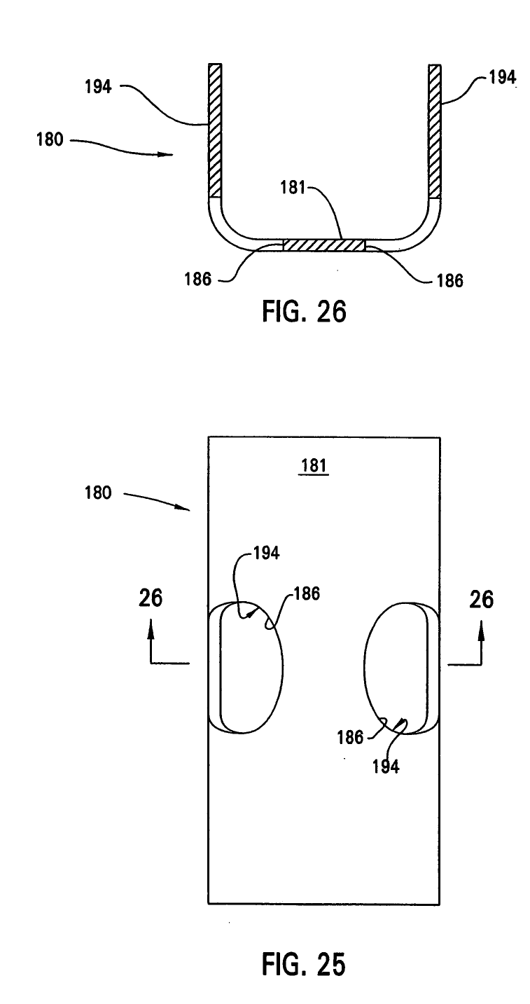

To complete the bridge 180, the sheet 190 is bent, or folded, along the dotted lines F to produce the generally U-shaped bridge 180 in FIGS. 25 and 26.

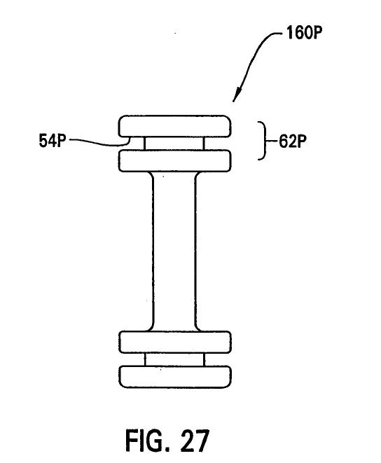

The blade 10P is preferably assembled as follows. With the blanks 40P and the bridge 180 bent as shown in FIG. 21, the bent blanks 40P are spaced opposite each other sufficient to allow insertion of the spacer end portions 62P into the opposed widened circular ends 54P of the keyhole slots 50P. The bent blanks 40P are then brought together slightly, sufficient that the edges of the keyhole slots 50 are coplanar with and engage the annular grooves 64P (FIG. 27) of the spacer 60P, whereafter the spacer 60P is moved distally along the keyhole slots 50P to its distal limiting position of FIGS. 19 and 21. Thus, due to the spacer 60P, the adjacent portions of the legs 30P cannot be moved together or apart, although the bent blanks 40P are otherwise somewhat moveable with respect to each other.

The bridge 180 is then inserted between the proximal portions of the blade legs, with its arms 191 extending proximally toward the angled intermediate portions 32P of the blade 10P and with the web 181 positioned as generally above discussed, mainly as seen in FIGS. 19 and 21. More particularly, the web 181 (which forms the bight of the U-shaped cross section of the bridge 180) is disposed substantially at the joinder of the widened circular end portions 54P and elongate narrow end portions 52P of the opposed keyhole slots 50P. The proximal portions of the bent blanks 40P are then brought together to their assembled position of FIGS. 19 and 21, snuggly sandwiching the arms 191 of the bridge 180 between the legs 30P and abutting the proximal portions of the bent blanks 40P which define the blade base 20P. Once in this position, the bent blanks 40P and bridge 180 can be permanently fixed together by any convenient means, as by laser spot welding, schematically indicated by the widthwise lines of dots SW1 (FIG. 17) of the blanks 40P immediately proximal of the divergently angled intermediate portions 32p thereof and by laser spot welding schematically indicated by the widthwise lines of dots SW2 (FIG. 17) of the bridge arms 191 to the flanking proximal portions of the blade legs 30P. That completes assembly of the blade 10P.

The above discussed advantages and operation of the FIG. 1-16 blades 10 and 10M apply as well to the blade 10P of FIGS. 17-27.

The blades 10 and 10M of FIGS. 1-16 have been found to perform well over a relatively large leg separation (space-between-legs) range (e.g. from 5 to 10 mm). However, applicants have noted that the residual holes 75A, 75B (FIG. 2), left by bending out the corresponding tabs, necessarily reduces the effective width and consequent total cross-sectional area of sheet material connecting the base 20 to the corresponding legs 30A, 30B, namely to the combined width and cross-sectional area of the longitudinally extending strap-like portions 73A, 73B flanking the holes 75A, 75B. The same is true of the residual tab holes 152A, 152B and 158A, 158B of the blade 10M of FIG. 13. Thus, the wider the tab 70B and hole 75B, the narrower the flanking straps 73B and vice versa.

The FIG. 1-16 embodiments have worked well, over a range of blade sizes. However, applicants have determined that, as compared to the FIG. 1-16 blades 10 and 10M, the FIG. 17-27 blade 10P works well even if leg length and spacing are further increased. Advantageously, such increased leg spacing enables cutting of wider slices of bone. Thus, blades 10P work well even at leg spacing (width of cut) exceeding about 13 mm. Thus, in the blade 10P, the web 181 (FIG. 22) bridges the space between the blade legs 30P without requiring the above discussed FIG. 1-16 holes 75A, 75B, etc. The web 181 preferably extends the full width of the adjacent portion of the blade 10P, thereby further rigidifying the blade 10p.

Moreover, the proximally extending arms 191 of the bridge 180 preferably extend the full width of the blade 10P to the longitudinal edges of the legs 30P, and about the proximal portions of the legs 30P over a substantial area. This makes it very easy to rigidly fix the bridge 180 to and between a substantial area of the legs 30P, as by laser spot welding the legs 30P to the arms 191. This strengthens the blade 10P against twisting and racking stresses, and thus against deflection of the legs 30A from normal parallel, mapped relation.

This strengthening effect is not significantly diminished by the through holes 194 (FIG. 123) in the central portion of the joinder of the web 181 and the arms 191, because the area of the holes is much smaller than that of the U-shaped bridge 180.

Moreover, the holes 194 are sized to receive the enlarged diameter end portions 62P of the dumbbell shaped spacer 60P to enable the reduced diameter central portion 66P of the spacer 60P to abut the web 181 of the bridge 180. Thus, the web 181 can be located as far forward, and hence as close to the cutting edges 34P of the legs 30P, as possible without diminishing the maximum depth of cut determined by the longitudinal spacing of the cutting edges 34P from the reduced diameter central portion 66P of the spacer 60P.

Moreover, only a relatively short portion (the tissue engaging portion) of the legs 30P is cantilevered forward from the bridge 180. Relatively short cantilevered portions better resist bending and twisting. This further reduces any tendency of the cutting edges 34P to distort from precise parallel and mapped relation, due to forceful twisting and bending stresses applied to the blade 10P as the surgeon forcefully moves the handpiece from side to side and/or by twisting same back and forth.

Moreover, the relatively short and stiff blade forward portions, which are cantilevered substantially from the maximum cutting depth position of the spacer 60P, their relatively low mass, as assisted by the lightening reliefs and holes therein, increases the resonant frequency of the blade 10P to allow use at even higher cutting speeds.

Moreover, the bridge arms 191 extend proximally from the web 181, substantially to the distal end of the divergent ramps 40P. The bridge thereby reinforces the portion of the legs 30P between the web 181 and ramps 40P, with the web 181 extending substantially perpendicular to said legs 30P and the bridge extending substantially the full width of the proximal portion of the legs 30P. The result is a rigid, reinforced, five sided box structure rigidly projecting proximally from the base 20P. This provides rigid support and positioning for cantilevered proximal portions of the blades 30P.

Moreover, in use, the blade's most massive part, comprising the mentioned box structure, is closer to the handpiece, so as to oscillate in a smaller radius, narrower arc, with lesser arcuate displacement and speed and lower end of stroke acceleration/deceleration, as compared to the relatively light and short, cantilevered proximal portions of the legs. In use, this minimizes the blade's ability to cause the operating handpiece to vibrate or jump in the surgeon's hand.

Moreover, in the assembled blade 10P, this forward positioning of the U-shaped member 180 enables the bridge web 181 to block escape of the spacer 60P through the widened circular slot ends 54P (FIGS. 19 and 20), at the proximal end of the keyhole slots 50P. As discussed above, this is achieved during assembly by first installing the spacer 60P and thereafter fixing in place the U-shaped bridge 180. This also advantageously allows the two blanks 40P of the blade 10P, to be identical to each other unlike in other known blades of this type. Also, by fixedly overlapping and bending the widened circular end portion 54P of the keyhole slot 50P, the bridge arms 191 effectively stiffen the legs against bending at the widened circular ends 54P of the keyhole slots 50P.

Moreover, the bridge 180 does not interfere with the provision of the concave lightening reliefs in the leg side edges 90P and lightening holes 92P.

Although particular preferred embodiments of the invention have been disclosed in detail for illustrative purposes, it will be recognized that variations or modifications of the disclosed apparatus, including the rearrangement of parts, lie within the scope of the present invention.

1. A surgical saw blade, comprising:
a surgical-power-tool-chuck-drivable base;
legs laterally opposed across a space, said legs being fixed with respect to and extending distally of said base and having substantially parallel, free, tissue working, distal end portions remote from said base;
an intermediate portion comprising ramps acutely angled divergently away from the plane of said base and fixedly connecting the distal end of said base to respective proximal portions of said legs.

2. The apparatus of claim 1 in which said ramps extend distally from said base to proximal ends of said legs in a substantially Y-like manner.

3. The apparatus of claim 1 in which said ramps angle proximally from the distal end of said base to respective proximal ends of said legs in a generally S-like manner.

4. The apparatus of claim 1 including a rigidifying bridge fixed with respect to and extending across the space between said legs adjacent said ramps, bridge being spaced proximally from said tissue working distal end portions.

5. The apparatus of claim 4 in which said ramps extend distally from said base to proximal ends of said legs in a generally Y-like manner, said ramps and bridge defining planes forming a generally triangular profile between said base and legs.

6. The apparatus of claim 4 in which said ramps angle proximally from the distal end of said base to respective proximal ends of said legs in a generally S-like manner, said ramps and legs and bridge forming at least one generally triangular profile, in which said bridge extends across said space fixedly from said legs and toward and into fixed connection with said base and ramps adjacent their distal ends, a further bridge having outer ends fixed to said ramps adjacent their proximal ends and an intermediate portion fixed to said base and proximally spaced from said distal ends of said ramps and base.

7. The apparatus of claim 4 in which said ramps diverge from the distal end portion of said base in a distal direction and in a substantially Y-like manner, said legs extending distally from distal end portions of said ramps, said bridge comprising a web fixed to and extending between said legs at a location spaced distally from said ramps.

8. The apparatus of claim 1 including material-removed, lightening portions on said legs distally of said ramps, said lightening portions being selected from the group consisting of concavities in longitudinal edges of said legs and through holes in the legs adjacent their distal end portions.

9. The apparatus of claim 1 in which said ramps diverge and include an angle in the range of 70-110 degrees.

10. A surgical saw blade, comprising:
a surgical-power-tool-chuck-drivable base;
a pair of legs laterally opposed across a space, said legs being fixed with respect to and extending distally of said base and having respective, substantially parallel free, tissue working distal end portions remote from said base;
a rigidifying bridge fixed with respect to and extending across the space between said legs, said bridge being spaced proximally from said tissue working distal end portions of said legs and rigidly locating said legs with respect to each other.

11. The apparatus of claim 10 including ramps extending distally from said base to proximal ends of said legs in a generally Y-like manner, said bridge being fixed adjacent distal end portions of said ramps to enclose a substantially triangle shaped space extending distally from said base to proximal end portions of said legs.

12. The apparatus of claim 10 including ramps angling proximally from the distal end of said base to respective proximal ends of said legs in an S-like manner, tabs extending inward toward each other from said legs and having inboard ends fixed with respect of each other adjacent the connected distal ends of said ramps and base, said ramps being opposed to the distal portion of the base, further tabs bent from proximal portions of said ramps and extending inboard into fixed engagement with said base and spaced proximally from the distal end of said base.

13. The apparatus of claim 10 in which said bridge comprises a web fixed with respect to and extending transversely between said legs, said web extending widthwise of said legs, ramps fixed to and extending divergently and distally from the distal end portion of said base to fixedly engage the proximal ends of said legs.

14. The apparatus of claim 13 in which said bridge is generally U-shaped and further comprises arms extending from said web, snuggly and reinforcingly along the opposed surfaces of said legs, substantially to the joinder of said ramps with said legs.

15. The apparatus of claim 14 in which said legs have opposed, longitudinally extending slots, and including a spacer having end portions slideably guided in such slots and moveable between longitudinally spaced locations on said legs, grooves in said spacer end portions slideably receiving portions of said legs defining opposite sides of said slots, said spacer having a central length portion extending between said spacer end portions and substantially spanning the space between said legs, said spacer end portions being enlarged with respect to said central length portion of said spacer, holes in said bridge at the joinder of said web and arms and receiving said spacer end portions at the proximal end of the path of said spacer along said slot with said spacer central length portion disposed immediately adjacent the distal face of said web, such that said bridge is located substantially as close as possible to the distal ends of said blades yet allowing the full desired (1) longitudinal extent of travel of said spacer along the length of said legs and (2) depth of cut of said blade.

16. The apparatus of claim 15 in which said longitudinal slots each have a widened proximal end portion of width greater than said spacer end portions and sufficient to receive said spacer longitudinally therethrough for mating said spacer and portion grooves slideably with the edges of the distal portion of said slots, said bridge substantially closing said widened proximal ends of said elongate slots to prevent escape of said spacer from said slots in the assembled blade and thereby allowing said base, ramps and legs to be formed by two laterally opposed, identical blanks.

17. The apparatus of claim 10 in which said bridge comprises a generally U-shaped member which includes a central web fixedly extending transversely between and substantially perpendicular to said legs at a location spaced distally from the proximal ends of said legs, and arms extending from said web snuggly and reinforcingly along opposed inboard surfaces of said legs, said bridge extending substantially the width of said legs, ramps fixed to and extending divergently and distally from the distal end portion of said base and fixedly engaging respective proximal ends of said legs at a location adjacent said arms and spaced proximally from said web, said legs having opposed longitudinally extending keyhole slots with elongate distal portions and widened proximal portions, a spacer slideably guided in such slots and moveable between longitudinally spaced locations on said legs, said spacer having end portions with faces slideably snuggly flanking outboard faces of said legs on opposite sides of said elongate distal portions, said spacer having a central length portion substantially spanning the space between said legs, said spacer end portions being widened with respect to said central length portion of said spacer, said slot proximal portions being wide enough to allow passage therethrough of at least one of said spacer end portions, holes in said bridge at the joinder of said web and arms and receiving said spacer ends at the proximal end of the path of said spacer along said grooves, said spacer central length portion being disposable immediately adjacent the distal face of said web, such that said bridge is located substantially as close as possible to the distal ends of said blades while yet allowing the full desired longitudinal travel of said spacer along the length of said legs, in which said spacer end portions span the thickness of said legs and have grooves receiving longitudinal edges of said slot distal portion.

18. A surgical saw blade, comprising:
a surgical-power-tool-chuck-drivable base;
substantially parallel legs laterally opposed across a space, said legs being fixed with respect to and extending distally of said base and having substantially parallel, free, tissue working, distal end portions remote from said base, said end portions of said legs comprising corresponding sets of cutting teeth, said legs having respective longitudinally extending slots;
a spacer having end portions slideably guided in said slots and moveable between longitudinally spaced locations on said legs, grooves in said spacer ended portions slideably receiving portions of said legs defining opposite sides of said slots, said spacer having a central length portion extending between said spacer end portions and substantially spanning the space between said legs, said spacer end portions being enlarged with respect to said central length portion of said spacer;
substantially bilaterally symmetrical, concave, semi-circular, lightening reliefs extending longitudinally in the side edges of each leg and extending from near the proximal end of the corresponding slot gradually inward toward the central portion of said slot and laterally outwardly gradually toward said leg distal end portion;
substantially bilaterally symmetrically located lightening holes through the distal portion of each said leg and having outward and distal sides respectively spaced close inboard from and extending along the distal portion of said concave reliefs and said distal edge of said leg, said lightening holes each having an inboard side spaced from the distal edge of said slot, said lightening holes reducing the mass of said leg adjacent said cutting teeth and remote from said base.

19. The apparatus of claim 18 including an intermediate portion comprising ramps acutely angled divergently away from the plane of said base and fixedly connecting the distal end of said base to respective proximal portions of said legs.

20. The apparatus of claim 18 including a rigidifying bridge fixed with respect to and extending across the space between said legs, said bridge being spaced proximally from said set of cutting teeth and rigidly locating said legs with respect to each other.
